(19) United States
(12) Patent Application Publication
(10) Pub. No.: US 2005/0124696 A1
(43) Pub. Date: Jun. 9, 2005

(54) Pharmaceutical compositions

(76) Inventors: Stanley Michael Roberts, Manchester (GB); Alan Michael Happe, Canterbury (GB); Maria Gabriella Santoro, Avellino (IT); Jerome Dauvergne, Chapel-en-le-Frith (GB); Herve Siandt, Huningue (FR)

Correspondence Address: JONES DAY, 222 EAST 41ST ST, NEW YORK, NY, 10017, US

(21) Appl. No.: 10/871,692

(22) Filed: Jun. 11, 2004

(30) Foreign Application Priority Data
Dec. 14, 2001 (GB) 0129979.1
Mar. 27, 2002 (GB) 0207232.0
Dec. 16, 2002 (WO) PCT/GB02/05708

(51) Int. Cl. A61K 31/198; C07C 323/30

(52) U.S. Cl. 514/562; 562/507

(57) ABSTRACT

Compounds of formulae (I) or (II): wherein R is a substituted or unsubstituted alkyl, alkenyl, alkynyl, aryl, aralkyl aralkenyl, or aralkynyl group, that optionally includes at least one heteroatom in its carbon skeleton, and R?1 [symbol] and R?2 [symbol] are H, or an —OR?3 [symbol] group in which R?3 [symbol] is a substituted or unsubstituted alkyl, alkenyl, alkynyl, aryl, aralkyl aralkenyl, or aralkynyl group containing 4-12 carbon atoms, that optionally includes at least one heteroatom in its carbon skeleton, and R?1 [symbol] and R?2 [symbol] cannot both be H, and their use in therapeutic methods.

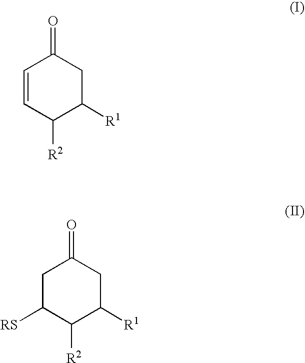

The present invention relates to certain cyclohexenone and cyclohexanone derivatives. It also relates to the preparation of such derivatives, and to their use in medicine and other fields. The invention further relates to certain cyclohexanone derivatives with enhanced water solubility and therapeutic indices, and to methods of enhancing the water solubility and therapeutic indices of pharmaceutically active cyclohexenone derivatives.

Various compounds comprising the cyclopentenone ring structure (also known as the cyclopentenone nucleus) are capable of inducing the heat shock response. The heat shock response is a finely regulated and highly conserved mechanism to protect cells against different types of injury, including extreme temperatures, oxidative stress, exposure to toxins and viral infection (1). In human cells, triggering of the heat shock response requires activation of a transregulatory protein, the heat shock transcription factor type 1 (HSF 1), which controls the expression of cytoprotective heat shock proteins (HSPs) (1). Whereas HSP induction was at first interpreted as a signal for detection of physiological stress, it is now accepted that HSPs are utilised by cells as molecular chaperones in the repair process following different types of injury to prevent damage resulting from the accumulation and aggregation of non-native proteins (1). In particular, a cytoprotective role of the heat shock protein HSP70 has now been described in a wide variety of human diseases, including ischemia, inflammation and viral infection (2-5). For these reasons HSF 1 is considered a novel, attractive target for cytoprotective and antiviral drugs. In the case of viral infection, Santoro et al. have shown that a class of prostaglandins (PGs) with potent antiviral activity function as HSP70 inducers via HSF1 activation (6,7).

The ability of prostaglandins of the A type (PGAs) to inhibit viral replication and prevent the establishment of persistent infections was first reported in 1980 (8). It is now well established that PGs containing an α,β-unsaturated carbonyl group in the cyclopentane ring structure (cyclopentenone PG, cyPG) possess activity against a wide variety of DNA and RNA viruses, including herpes viruses, paramyxo viruses, orthomyxo viruses and retroviruses in in vitro and in vivo experimental models (9). The mechanism of the antiviral activity is distinct from any other known antiviral agent and is thought to involve the induction of heat shock proteins and the inhibition of the transcription factor NF-κB (nuclear factor-κB) in the infected cell.

NF-κB is an inducible eukaryotic transcription factor which has a critical role in promoting inflammation and viral replication (11). In most cells, NF-κB exists in an inactive cytoplasmic complex, whose predominant form is a heterodimer composed of p50 and p65 subunits, bound to inhibitory proteins of the IκB family, usually IκBα, and is activated in response to primary (viruses, bacteria, UV) or secondary (inflammatory cytokines) pathogenic stimuli (12). Stimulation triggers rapid phosphorylation and degradation of IκBΕ, resulting in NF-κB translocation to the nucleus, where the factor binds to DNA at specific κB-sites, inducing a variety of genes encoding signalling proteins. Target genes include those coding for inflammatory and chemotactic cytolines, cytokine receptors and viral proteins. NF-κB is involved in many pathological events including progression of AIDS by enhancing HIV-1 transcription and is considered an attractive therapeutic target for novel antiviral and anti-inflammatory drugs (12). Santoro et al. have shown that cyclopentenone prostaglandins inhibit NF-κB activation and NF-κB dependent HIV-1 transcription in human cells, by preventing IκBα phosphorylation and degradation, and that this effect is strictly associated with HSF1 activation (11).

Santoro et al. have identified the molecular structure of natural prostaglandins responsible for HSF activation and NF-κB inhibition (13). One component of the PGA molecule, cyclopent-2-en-1-one (also known as 2-cyclopenten-1-one), at a concentration of 125-500 μM, has been shown to be able to activate-HSF1 and to rapidly and selectively trigger the synthesis of cytoprotective HSP70. At the same concentration, cyclopent-2-en-1-one has been shown to be able to block NF-κB activation by chemical or physiological inducers. These effects are associated with antiviral activity during infection with rhabdoviruses (13).

There is no disclosure in the literature of the exhibition of any similar biological activity by the cyclohexenone analogues of the above discussed cyclopentenone derivatives.

Surprisingly, it has now been found that certain cyclohexenone and cyclohexanone derivatives are pharmaceutically active in at least one of the aforementioned ways.

According to a first aspect of the present invention, there is provided a compound of formula I or II: 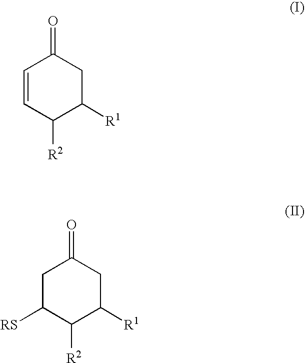
wherein:-
R is a substituted or unsubstituted alkyl, alkenyl, alkynyl, aryl, aralkyl aralkenyl, or aralkynyl group, that optionally includes at least one heteroatom in its carbon skeleton; and,
R¹ and R² are H, or an —OR³ group in which R³ is a substituted or unsubstituted alkyl, alkenyl, alkynyl, aryl, aralkyl aralkenyl, or aralkynyl group containing 4-12 carbon atoms, that optionally includes at least one heteroatom in its carbon skeleton, and R¹ and R² cannot both be H.

R can be an R⁴CH₂— group, wherein R⁴ is a substituted or unsubstituted alkyl, alkenyl, alkynyl, aryl, aralkyl aralkenyl, or aralkynyl group, that optionally includes at least one heteroatom in its carbon skeleton. R, preferably, contains 1-12 carbon atoms.

In preferred embodiments, R includes at least one hydrophilic group. Said hydrophilic group can be or include a hydroxyl, carbonyl, carboxyl, amino, amido, quaternary ammonium or thiolyl group. R, therefore, can provide the functionality of an amine, amide, peptide, ester, carboxylic acid, carboxylic acid salt, alcohol, aldehyde, ketone or thiol to an inventive compound.

In further preferred embodiments, the group —SR is an S-cysteinyl or a substituted S-cysteinyl group. In the context of this application, a substituted or unsubstituted S-cysteinyl group comprises a cysteinyl moiety that is bound to the ring via its sulphur atom, with the ring replacing the hydrogen atom that is bound to the equivalent sulphur atom in cysteine. Preferred substituted S-cysteinyl groups include di- and tri-peptide groups that include an S-cysteinyl moiety, such as S-glutathionyl, S-cysteinyl ester and other like groups, including N-tert-butoxycarbonyl S-cysteinyl and N-tert-butoxycarbonyl S-cysteinyl ester (e.g. methyl and ethyl) groups.

Preferably, only one of R¹ and R² is an —OR³ group, in which R³ is previously defined, and the other is H. In all aspects of the invention, R³ is preferably an alkyl group that includes a heteroatom in its carbon skeleton. The heteroatom is preferably silicon and, in preferred embodiments, R³ is a trialkylsilyl group, preferably a tert-butyldimethylsilyl group.

Certain compounds in accordance with the invention can exist in the form of a least two enantiomers and all such enantiomers, unequal mixtures thereof and racemates, are encompassed by the present invention. Both R- and S-enantiomers of these compounds are useful. They can each be provided in a form substantially free of the other enantiomer (e.g. at least 75%, 85%, 90%, 95% or 99% free (w/w)). Mixtures of enantiomers (e.g. racemic mixtures) may however also be used.

Many compounds in accordance with the invention exist in both cis- and trans-forms, i. e. with R¹ and R² being cis- or trans- to each other in the cyclohexanone or cyclohexenone ring. The present invention encompasses all such individual isomers and mixtures thereof, together with their uses.

Preferred compounds in accordance with the first aspect of present invention include the following:-

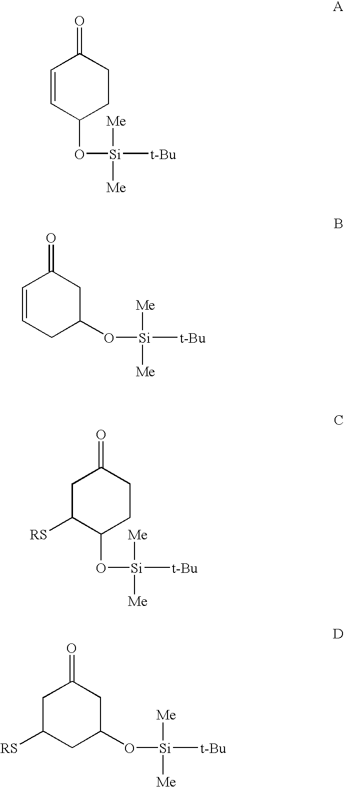

In the foregoing formulae, R is as defined above.

In an alternative aspect, the present invention provides a compound of formula I or II, as defined above, wherein said compound is not a compound of formula A or B. Preferred compounds in accordance with this alternative aspect of the invention include compounds of formulae C and D.

Compounds in accordance with the invention may be prepared by the techniques described in the examples. In particular, compounds that include the group RS—, such as compounds C and D, may be prepared from their cyclohex-2-en-1-one analogues by a technique of the type described in general method A (see below). The required cylcohex-2-en-1-one analogues Can be prepared by a technique of the nature described in Examples 1 and 2.

Compounds in accordance with the invention preferably are capable of one or more of the following:
a) activating HSF
b) inhibiting NF-κB
c) inhibiting the replication of BSV-1
d) inhibiting the replication of Sendai virus.

A skilled person can readily assay for the above activities and examples of suitable assays are set out in Examples 4, 5 and 7 below.

Compounds that have greater activity in at least one of the foregoing respects than cyclopent-2-en-1-one (at least at certain concentrations) represent preferred embodiments of the invention; those that enjoy such activity at a concentration within the range of 1-200 μM, or over the whole or a part of said range, being particularly preferred. Preferably, compounds in accordance with the invention have a level of activity in at least one of the foregoing respects that is at least twice the level of cyclopent-2-en-1-one. More preferably, it is at least 10 times that of cyclopent-2-en-1-one.

Activity in one of the above respects is indicative of a compound's capacity to be pharmaceutically active. Accordingly, in a yet further aspect, the present invention provides a compound in accordance with the invention for use in medicine (including veterinary medicine). Preferred such uses include the treatment of the human or animal body by therapy and diagnostic methods practised upon the human or animal body. The treatment may be prophylactic or may be in respect of an existing condition. Therapeutic (including prophylactic) and diagnostic methods, involving the use of a compound in accordance with the invention, are also within the remit of the invention.

The use of such compounds for the manufacture of medicaments for use in therapeutic or diagnostic methods to be practised on the human or animal body, lie within the scope of a further aspect of the invention.

The preferred uses for compounds in accordance with the invention include the treatment of disorders which can be treated in a host by the activation of a heat shock transcription factor (e.g. HSF1), by the induction of heat shock proteins (e.g. hsp70) and/or by the inhibition of NF-κB. Certain preferred compounds in accordance with the invention can be used in therapeutic applications that involve activating HSF and inhibiting the activity of NF-κB.

Thus, in accordance with the invention, compounds in accordance with the invention can be used to treat diseases or conditions in which such activity is indicated or can be of advantage. They can also be used in the manufacture of medicaments for use in such treatments. The preferred therapeutic and diagnostic applications for the inventive compounds are discussed in detail below.

Many pharmaceutically active compounds are poorly soluble in water or highly lipophilic. Such compounds are less suited, therefore, to being administered to patients orally than by other routes of administration, that are generally less favoured by patients, such as by parenteral injection.

The therapeutic index of a drug or pharmaceutically active compound is indicated by the ratio of its median lethal dose, or LD₅₀ to its medium effective dose, or ED₅₀. Because its use would generally involve a lower risk of causing toxic side effects in individual patients given a therapeutically effective dose, a compound with a larger therapeutic index would normally be preferred over an alternative with a smaller therapeutic index. Accordingly, if the therapeutic index of a particular pharmaceutically active compound could be increased without ill effect, this would be an attractive result.

Preferred compounds of formula II are:-
(a) more soluble in water at a temperature of 20-40° C.;
(b) less lipophilic; and/or,
(c) have a greater therapeutic index;
   than equivalent compounds of formula I. An equivalent compound of formula I to a preferred compound of formula II is a compound with, excepting the absent —SR group and an additional hydrogen atom in the 2 position in the six membered ring, the same substitution pattern as the preferred compound of formula II.

Where a preferred compound in accordance with the invention is required to be less lipophilic than an “equivalent” compound, this means that the ratio of n-octanol to aqueous solubility (i.e. the n-octanol/water partition coefficient) for the preferred compound is lower than it is for the “equivalent” compound. This ratio is usually expressed in terms of its base ten logarithm, “logP”, and a compound with a logP value of 1 will be 10 times more soluble in n-octanol than it is in water, a compound with a logP value of 2 will be 100 times more soluble in n-octanol than it is in water and so on. LogP values can be measured by experiment, or calculated using one of several available computer programs or algorithms. Examples of these include the Pomona College Medicinal Chemistry program and the method described by Moriguchi et al.(20). Thus, it is preferred that compounds, required in this specification to be more lipophilic than equivalent compounds, will have lower logP values than such equivalents. In this context, the logP values are preferably calculated values derived from applying one of the aforementioned programs or algorithms.

Where a preferred compound in accordance with the invention is required to have a greater therapeutic index than an “equivalent”, this relationship must hold true for at least one therapeutic application. For the purposes of this specification, the existence of such a relationship can be established either by observation of in vivo effects, or via in vitro tests or assays of the type conventionally employed by persons skilled in the art for the purpose of predicting the therapeutic indices of putative drug substances. For example, an assay for one of the properties discussed below could be used in combination with a toxicity assay, to provide the required information for a particular pair of inventive compound and equivalent. Examples of appropriate assays are set out in Examples 4-8 below.

In preferred embodiments, the preferred compounds of formulae II will have a calculated or measured logP value that is at least 0.25, 0.5, 0.75, 1 or 1.25 lower than the logP value for their equivalents of formula Ii wherein the logP values for the compounds are calculated or measured using the same technique. Preferably, the preferred compounds of formula 11 have a logP value of 5 or less, and preferably of 4.15 or less when calculated by the method described by Moriguchi et al. (20). Compounds with logP values in these latter preferred ranges are generally more readily absorbed from the gastro-intestinal tract and, therefore, are more suited to oral administration. See Lipinski et al. (21).

An advantage of the preferred compounds of formula II are that, because they are less lipophilic and/or more soluble in water at around room temperature and/or body temperature than are their equivalents of formula I, that do not include an -SR substituent, they are more suited to use in orally administered pharmaceutical compositions. Moreover, because such compounds of formula II can also have a greater therapeutic index than their equivalents without an —SR substituent, they are potentially more useful in a therapeutic context.

Cyclopentenone compounds are known to undergo Michael reactions with glutathione in the cell. Glutathione is found throughout the body and plays crucial roles in protecting cells from oxidative damage (maintaining redox balance). In this regard, work by Uchida et al. (22) and others has suggested a role for glutathione in protecting cells from oxidative stress as a radical scavenger. Uchida's work showed that cells with depleted glutathione retain higher concentration of radical oxygen species. It also demonstrated that, when such cells were treated with N-acetyl-cysteine and cell viability was measured, an increase in cell life and a decrease in the production of radical oxygen species was observed. Uchida et al. came to the conclusion that species capable of reducing glutathione levels in the cell, also reduce the cell's anti-oxidant defences and increase the induction of radical oxygen species. They also showed that cyclopentenone mediated production of radical oxygen species was well correlated with cytotoxicity and, thus, demonstrated a potentially important mode of cytotoxicity or induction of cell death by cyclopentenone compounds.

Glutathione is also known to protect cells from dangerous-electrophilic species. For example, morphine type compounds undergoes a Michael reaction with glutathione that results in complete deactivation of the drug (23). If large amounts of paracetamol (acetaminophen) are taken then glutathione in the liver is depleted [in 1999 there were 150 deaths in the UK from paracetamol poisoning]. If N-acetyl cysteine is taken intravenously or orally less than 15 h after the overdose it effectively removes the offending electrophilic paracetamol metabolite (24).

Other studies have shown that a reduction of intracellular thiol content can increase the sensitivity of tumour cells to radiation treatment. Moreover, cells exhibiting depleted levels of glutathione have been shown to be more susceptible to radiation, chemotherapeutic agents and oxygen radical species that otherwise would have been destroyed via radical reaction with glutathione (25).

A glutathione group cannot be added to a saturated moiety, such as a cyclohexanone group, via a Michael reaction. Thus, unless they are metabolised into the equivalent unsaturated cyclohex-2-en-1-ones, compounds in accordance with the invention that comprise a cyclohexanone group may be less likely to react with glutathione in vivo than are these unsaturated equivalents. Such saturated compounds, therefore, may be less likely to deplete the levels of glutathione in a patient's cells, and thereby compromise their anti-oxidant defences, than the equivalent cyclohex-2-en-1-one derivatives. Without wishing to be bound by theory, this may explain why some compounds in accordance with the invention that include an —SR group have significantly enhanced therapeutic indices, in addition to enhanced water solubility and reduced lipophilicity.

Without again wishing to be bound by theory, it is considered that compounds in accordance with the present invention, wherein the carbon atom in the 3 position in their cyclohexanone rings carries an —SR group, enjoy their enhanced properties partially because they can act as pro-drugs for the equivalent cyclohex-2-en-1-ones, in the sense that it is thought that they are converted into the latter in vivo. In this regard, it is considered that, before it is cleaved, the group —SR renders these compounds more water soluble and less lipophilic than their equivalents, and hence more suited to oral delivery, and that in vivo cleavage of the —SR group releases, via a reverse Michael reaction, the more potent cyclohex-2-en-1-one equivalent. Thus, in embodiments, compounds of formula II in accordance with the invention are transformable into equivalent cyclohex-2-en-1-one derivatives of formula I by a reverse Michael reaction, or are pro-drugs for such equivalents.

In further preferred embodiments, the group —SR is an S-cysteinyl or a substituted S-cysteinyl group. Preferred substituted S-cysteinyl groups include di- and tri-peptide groups that include an S-cysteinyl moiety, such as S-glutathionyl, S-cysteinyl ester and other like groups, including N-tert-butoxycarbonyl S-cysteinyl and N-tert-butoxycarbonyl S-cysteinyl ester (e.g. methyl and ethyl) groups

Without once again wishing to be bound by theory, it is considered that compounds in accordance with these latter embodiments of the invention are also capable of providing a secondary therapeutic effect resulting from their incorporation of a substituted or unsubstituted cysteinyl moiety. For example, when acting as pro-drugs in the aforementioned manner, such compounds may be capable of delivering both the equivalent cyclohex-2-en-1-one derivative and the reduced form of the substituted or unsubstituted cysteinyl moiety to target cells in a patient's body, where both may exert their therapeutic effects. The therapeutic effect exerted by the reduced form of the substituted or unsubstituted cysteinyl moiety can be the prevention of glutathione depletion, especially when the reduced moiety is glutathione, an analogue or precursor. For example, the reduced, substituted or unsubstituted cysteinyl moiety may compete with native glutathione, to reduce the amount of the latter that is bound by the cyclohex-2-en-1-one derivative (formed after in vivo cleavage) or a metabolite, or it may replace or lead to the replacement of glutathione bound by the derivative or a metabolite. Such activity is thought to contribute significantly to the reducing the toxicity of the inventive compounds and, hence, to the increased therapeutic indices enjoyed by these compounds, in comparison to the equivalent cyclohex-2-en-1-one.

In a further embodiment of the present invention, there is provided a method of decreasing the lipophilicity and/or increasing the water solubility and/or the therapeutic index of a pharmaceutically active compound of formula I as defined above, said method comprising forming an adduct of said compound of formula I and a thiol of the formula HSR, wherein R is as herein before defined and the adduct is of formula II, as defined above.

The adduct may act as a pro-drug in the manner discussed above, or it may be pharmaceutically active in its own right.

In preferred embodiments of this method, the adduct is formed via a Michael reaction between the unsaturated compound of formula I and the thiol. A preferred method of forming the adduct is described in the examples that follow.

In a further aspect, the present invention provides an adduct as herein before defined, prepared or preparable by a method in accordance with the invention.

For the avoidance of doubt, it is confirmed that the term “alkenyl” denotes an a group with one or more double bonds in its carbon skeleton and the term “alkynyl” denotes a group with one or more triple bonds in its carbon skeleton. It should also be understood that, for the purposes of this specification, alkynyl groups may include both double and single bonds in their carbon skeletons. Unless otherwise specified, groups referred to in this specification as alkyl, alkenyl or alkynyl groups can be straight chained or branched, or be or include cyclic groups. However, unless the contrary is indicated, they are preferably straight chained or branched.

Medical uses for compounds in accordance with the invention The preferred uses for compounds in accordance with the invention include the treatment of disorders which can be treated in a host by the activation of a heat shock transcription factor (e.g. HSF1), by the induction of heat shock proteins (e.g. hsp70) and/or by the inhibition of NF-κB.

Certain preferred compounds in accordance with the invention can be used in therapeutic applications that involve activating HSF and inhibiting the activity of NF-κB. Thus, in accordance with the invention, such compounds can be used to treat diseases or conditions in which such activity is indicated or can be of advantage. They can also be used in the manufacture of medicaments for use in such treatments. Preferred therapeutic and diagnostic applications for such compounds are discussed below.

It should be appreciated that certain compounds in accordance with the invention do not exhibit activity in all of the respects discussed above. Such compounds, therefore, may only find use in those of the therapeutic and diagnostic applications detailed below where their properties are indicative of potential usefulness.

It should be appreciated that certain disorders, e.g. cancers, may be mediated by viruses and by non-viral factors. In the absence of any indication to the contrary, treatment of any given disorder is covered whether or not the disorder is mediated by viruses. It should also be appreciated that there is some overlap between the various categories of treatment discussed, i.e. the categories are not intended to be mutually exclusive.

1. Treatment of Viral-Mediated Disorders

NF-κB is implicated in the pathogenesis of certain viral infections. It is known that heat shock proteins (e.g. HSP70) can offer protection against the pathogenesis of viral infection. Compounds in accordance with the invention may be active in reducing the replication of viruses.

Compounds in accordance with the invention may be useful in treating viral-mediated disorders. These include disorders mediated by RNA viruses, as well as disorders mediated by DNA viruses.

Examples of viral disorders that may be treated using compounds in accordance with the invention include the following.

Diseases caused by or associated with members of the Adenoviridae family, including (but not limited to): diarrhea or intussusception caused by or associated with enteric adenoviruses, upper or lower respiratory tract infections (including the common cold or pneumonia) caused by or associated with respiratory adenoviruses; conjunctivitis, keratitis or trachoma caused by or associated with adenovirus infection of the eye; tonsillar or kidney infections caused by or associated with adenoviruses.

Diseases caused by or associated with members of the Arenaviridae family, including (but not limited to): Lassa fever caused by Lassa fever virus; meningitis caused by or associated with lymphocytic choriomeningitis virus; hemorrhagic fevers including (but not limited to) those caused by Machupo virus, Junin virus, Sabia virus, Guanarito virus or Tacaribe virus.

Diseases caused by or associated with members of the Astroviridae family, including (but not limited to): diarrhea caused by or associated with astroviruses.

Diseases caused by or associated with members of the Bunyaviridae family, including (but not limited to): hemorrhagic fever with renal syndrome, hantavirus pulmonary syndrome, or other diseases caused by or associated with hantaviruses including (but not limited to) Hantaan virus, Puumala virus, Seoul virus, Dobrava virus, Sin Nombre virus, bayou virus, Black Creek canal virus, New York 1 virus, Monogahela virus, Andes virus, Laguna Negra virus; arbovirus infections including (but not limited to) La Crosse encephalitis, California encephalitis, or other bunyavirus infections; Rift Valley fever, sandfly fever, Uukuniemi or other arbovirus infections associated with phleboviruses; Crimean-Congo hemorrhagic fever or other infections caused by Nairoviruses.

Diseases caused by or associated with members of the Caliciviridae family or related agents, including (but not limited to): hepatitis caused by or associated with hepatitis E virus, diarrhea caused by or associated with caliciviruses or small round structured viruses.

Diseases caused by or associated with members of the Coronaviridae family, including (but not limited to): lower or upper respiratory tract infections (including the common cold) caused by or associated with coronaviruses; diarrhea, enterbcolitis or gastroenteritis caused by or associated with coronaviruses or toroviruses.

Diseases caused by or associated with members of the Filoviridae family, including (but not limited to): hemorrhagic fevers caused by Ebola or Marburg viruses.

Diseases caused by or associated with members of the Flaviviridae family, including (but not limited to): arbovirus infections, fevers or encephalitides including (but not limited to) yellow fever, Kyansur Forest disease, Omsk hemorrhagic fever, other tick-borne encephalitis infections. Rocio, Japanese encephalitis, St. Louis encephalitis, West Nile virus infection, Murray Valley encephalitis, Dengue fever, or Dengue hemorrhagic fever caused by or associated with flaviviruses; hepatitis caused by or associated with hepatitis C virus.

Diseases caused by or associated with members of the Hepadnaviridae family, including (but not limited to): hepatitis caused by or associated with hepatitis B virus.

Diseases caused by or associated with members of the Herpesviridae family, including (but not limited to): orolabial herpes, genital herpes, herpetic dermatitis, herpetic whitlow, zosteriform herpes simplex, ocular disease, encephalitis or neonatal herpes caused by or associated with herpes simplex viruses types 1 or 2; chickenpox, shingles, zoster-associated pain, pneumonia, encephalitis, fetal infection or retinal necrosis caused by or associated with varicella-zoster virus; transplant rejection, congenital infection, infectious mononucleosis, retinitis or other diseases of the immunocompromised caused by or associated with cytomegalovirus; infectious mononucleosis, lymphomas, carcinomas or other cancers caused by or associated with Epstein-Barr virus; exanthem subitum, roseola infantum, pneumonitis or hepatitis caused by or associated with human herpesviruses 6 or 7; Kaposi's sarcoma or other neoplastic disease caused by or associated with human herpesvirus 8 (KSV).

Diseases caused by or associated with members of Orthomyxoviridae family, including (but not limited to): influenza, pneumonia, other respiratory infections, myositis, myoglobinuria, or Reye's syndrome caused by or associated with influenza viruses A, B or C.

Diseases caused by or associated with members of the Papovaviridae family, including (but not limited to): papillomas, comdylomas, neoplasias and carcinomas caused by or associated with papillomaviruses diseases caused by BKV or JCV viruses; progressive multifocal leukoencephalopathy caused by polyomaviruses.

Diseases caused by or associated with members of the Parvoriridae family, including (but not limited to): anemia, fever, fetal infection or hepatitis caused by or associated with parvorvirus B19.

Diseases caused by or associated with members of the Paramyxoviridae family, including (but not limited to): pneumonia, bronchiolitis, tracheobronchitis or croup caused by or associated with parainfluenza viruses; bronchiolitis or pneumonia caused by or associated with respiratory syncytial virus; encephalitis, measles or complications of measles including (but not limited to) pneumonia or sub-acute sclerosing panencephalitis (SSPE) caused by or associated with measles virus; mumps or complications of mumps including (but not limited to) orchitis or pancreatitis caused by or associated with mumps virus.

Diseases caused by or associated with members of the Picornaviridae family, including (but not limited to): hepatitis caused by or associated with hepatitis A virus; upper respiratory tract infections (including the common cold) caused by or associated with rhinoviruses or other respiratory picornaviruses; poliomyelitis caused by polioviruses; Bornholm disease, encephalitis, meningitis, herpangina, myocarditis, neonatal disease, pancreatitis, fever, conjunctivitis, chronic fatigue syndrome (ME) or hand, foot and mouth disease caused by coxsackieviruses or enteroviruses.

Diseases caused by or associated with members of the Poxviridae family, including (but not limited to): smallpox caused by smallpox virus; human forms of monkeypox or cowpox virus infections; infections with vaccinia virus including (but not limited to) complications of vaccination; orf or paravaccinia caused by parapoxviruses; molluscum contagiosum caused by molluscipoxviruses; infections with Tanapox virus.

Diseases caused by or associated with members of the Reoviridae family, including (but not limited to): diarrhea caused by or associated with rotaviruses.

Diseases caused by or associated with members of the Retroviridae family, including (but not limited to): acquired immune deficiency syndrome and associated disorders caused by or associated with human immunodeficiency virus (HIV); leukaemias, lymphomas, or myelopathies caused by or associated with HTLV viruses.

Diseases caused by or associated with members of the Rhabdopiviridae family, including (but not limited to): rabies caused by rabies virus; other lyssavirus diseases including (but not limited to) those caused by Duvenhage or Mokola viruses.

Diseases caused by or associated with members of the Togoaviridae family, including (but not limited to): rubella or congenital rubella syndrome caused by rubella virus; fever or encephalitis caused by eastern equine encephalitis virus, Venezuelan equine encephalitis virus, western equine encephalitis virus, Everglades virus or Semliki Forest virus; fever, rash, polyarthritis, myalgia or arthralgia caused by Sindbis virus, Ockelbo virus, Ross River virus, Barmah Forest virus, Chikungunya virus, O∝nyong-nyong virus, Mayaro virus or Igo Ora virus.

Diseases caused by or associated with viroid-like agents, including (but not limited to): hepatitis caused by or associated with the delta agent (HDV).

Diseases caused by or associated with prions, including (but not limited to): Creutzfeld-Jakob disease (CJD), new variant CJD, GSS, and fatal familial insomnia.

Compounds of the present invention may be particularly useful in treating viral and other disorders affecting aquatic organisms (e.g. fish, crustaceans, etc.). Such disorders include disorders mediated by the snout ulcer virus, by the iridovirus, by the lymphocystis disease virus, etc.

Compounds in accordance with the invention may therefore be used in aquaculture. They may be used in food for aquatic organisms. Such food is within the scope of the present invention. It will generally be sold in sealed containers and labelled appropriately (e.g. as fish food, food for crustaceans, food for aquatic organisms, etc.). Alternatively, compounds in accordance with the invention may be used for water treatment or for direct application to aquatic organisms. Such compounds do not therefore need to be present in foodstuffs in order to be useful in aquaculture.

2. Treatment of Bacterial-Mediated Disorders

NF-κB is activated in response to bacterial infections.

Compounds in accordance with the invention can be useful in treating disorders arising from such infections, e.g. in treating NF-κB stimulated inflammation. Most commonly this will arise due to infection with gram negative bacteria. However it may also arise due to infection with gram positive bacteria (e.g. S. areus).

3. Treatment of Disorders Mediated by Radiation

NF-κB is activated in response to radiation (e.g. UV-radiation).

Compounds in accordance with the invention can be useful in treating disorders mediated by radiation. Such disorders include cell and tissue trauma, cell and tissue ageing and cancer (e.g. skin cancer).

4. Treatment of Inflammation and of Disorders of the Immune System

NF-κB is activated in response to inflammatory cytokines. It is believed to be an early mediator of the immune and inflammatory responses.

Compounds in accordance with the invention can be useful in treating immune disorders (e.g. auto-immune disorders) and in treating inflammatory disorders. Examples of specific inflammatory disorders and disorders of the immune system that may be treated with such compounds include psoriasis, rheumatoid arthritis, multiple sclerosis, adult respiratory distress syndrome, hepatitis and/or cirrhosis, vascular inflammation (including lupus exythematosis disseminata), and inflammatory disorders of the gastro-intestinal tract (e.g. ulcers).

5. Treatment of Ischemia and Arteiosclerosis

NF-κB has been implicated in the pathogenesis of ischemia and anteriosclerosis. Compounds in accordance with the invention are therefore useful in treating such disorders, including reperfusion damage (e.g. in the heart or brain) and cardiac hypertrophy.

6. Treatment of Disorders Involving Cell Proliferation

NF-κB is implicated in cell proliferation.

Compounds in accordance with the invention can be useful as anti-proliferatives. They are therefore useful in treating-inflammatory granulomas, neointimal proliferation in arterial and venous restenosis, and cancers (including lymphomas, leukemias, sarcomas, carcinomas and melanomas).

7. Treatment of Disorders Involving Damage to or Killing of Cells

Heat shock proteins are known to provide a cytoprotective effect.

Compounds in accordance with the invention can be useful in treating disorders involving damage to or killing of cells.

These disorders include chemical toxicity (e.g. due to ingestion of toxins, such as paraquat, or to overdosing with medicaments, such as paracetamol), oxidative cell damage, cell and tissue ageing trauma, hepatitis diabetes and the effect of burns. The inventive compounds, also, can be used to combat the effects of ageing in a human or animal, and to promote wound healing.

Other conditions of this general nature, that can be treated using compounds of the present invention, include oxidative stress and degenerative diseases, especially neuro-degenerative diseases such as BSE, new variant CJD and Alzheimer's disease.

8. Other Treatments

Cyclopentenone prostaglandins are of known utility in stimulating peroxisome proliferator activated receptors (PPARs). Compounds in accordance with the invention, thus, can be useful in treating diabetes (including complications arising therefrom). Such compounds can also be used in the treatment of disorders in which calcium loss or deficiency is implicated or involved (including bone disorders, skeletal disorders, dental disorders, developmental disorders, etc.)

9. Treatments Employing HSF Selective Compounds

Compounds in accordance with the present invention can exhibit a capacity to trigger a heat shock response, activate HSF, or induce HSP expression, at a concentration at which they have no significant inhibitory effect on NF-κB activity.

In the light of the reports discussed in the opening paragraphs of this specification (see references 6, 7, 11 and 13), suggesting that compounds that have a capacity to activate HSF will also inhibit the activity of NF-κB, the selective action of compounds in accordance with the invention is highly surprising. This unexpected property, however, renders these compounds uniquely useful in therapeutic applications where an effect upon the heat shock response is desirable, but any interruption of the normal NF-κB pathway would be unnecessary, undesirable or possibly deleterious. For example, because the NF-κB pathway plays an important role in T-cell mediated immune responses, its interruption could be immunosuppressive and, therefore, unless required in order to achieve a particular therapeutic objective, in principle should be avoided. Thus, these compounds can be particularly useful in the treatment of viral infections in which the pathology of the virus does not involve an inflammatory component, or in which the killing of cells by the virus is more important in the pathology than is any inflammatory response. Such viruses include those that do not depend upon NF-κB for their replication or do not have κB elements in their genomes. In addition to viral infections, HSF selective compounds can be used to treat other conditions which do not involve an inflammatory component, and they are particularly useful in cytoprotective applications.

Their selectivity allows HSF selective compound to be used in situations where it is desirable for an NF-κB mediated inflammatory immune response to be maintained. For example, they are especially useful in chronic or prophylactic treatments, as long term suppression of NF-κB activity and, consequently, of a patient's full immune response to infection, can lead to unwanted opportunistic infections. It is also known that long term suppression of NF-κB activity can cause apoptosis in the liver.

Thus, the HSF selective compounds in accordance with the invention can be used in therapeutic applications that involve activating HSF without significantly inhibiting the activity of NF-κB. Therefore, in accordance with the invention, these compounds can be used to treat diseases or conditions in which such activity is indicated or can be of advantage. They can also be used in the manufacture of medicaments for use in such treatments.

Heat shock proteins are known to provide a cytoprotective effect. Thus, HSF selective compounds can be useful in cytoprotective applications and in treating (including by prophylaxis) disorders involving damage to or killing of cells.

These disorders include chemical toxicity (e.g. due to ingestion of toxins, such as paraquat, or to overdosing with medicaments, such as paracetamol), oxidative cell damage, cell and tissue ageing trauma, hepatitis, diabetes and the effect of burns. These compounds, also, can be used to combat the effects of ageing in a human or animal, and to promote wound healing.

Other conditions of this general nature, that can be treated using HSF selective compounds, include oxidative stress and degenerative diseases, especially neuro-degenerative diseases such as BSE, new variant CJD and Alzheimer's disease.

The cytoprotective effect of HSF selective compounds also tenders them useful in the treatment of ischemia and the damage resulting from episodes of ischemia and subsequent reperfusion. They can be employed to ameliorate the damaging effects of radiation and/or chemotherapy particularly, but not exclusively, when used in the treatment of cancer. These compounds can also be used to eat certain types of ulcers within the gastrointestinal tract.

As suggested in a foregoing section, compounds in accordance with the invention can be used as anti-viral agents. BSF selective compounds are useful, in general, in the treatment of viral infections wherein the pathological effects of the infecting virus can be reversed or prevented by a heat shock response. In particular, they can be employed to treat viral infections in which an inflammatory component is not significantly involved in or essential to the pathology of the infecting virus, the pathology of the virus does not involve an inflammatory component, or the killing of cells by the virus is more important than any inflammatory response. Such viruses include those that are not dependant upon NF-κB for their replication, or do not have κB elements in their genomes. Examples include parvoviruses, rotaviruses and those that infect the upper respiratory tract, including picornaviruses, coronaviruses and adenoviruses.

HSF selective compounds can also be used to treat infection with certain viruses that involve NF-κB and inflammation in their pathology, as the effects of many such organisms are reversed or prevented by the heat shock response and there may be other reasons why it may not be appropriate to administer an agent that disrupts the NF-κB pathway to a particular patient.

Examples of viral infections that can be treated with HSF selective compounds include infections with Picornaviruses (including Rhinoviruses and Hepatitis A virus), Reoviruses (including Rotavirus), Parvoviruses, Paramyxoviruses (including Sendai virus), Rhabdoviruses (e.g. vesicular stomatitis virus and rabies viruses), Filoviruses (e.g. Ebola virus), Adenovirus and Coronavirus. Viral infections with pathologies that involve inflammation and the NF-κB pathway, but which can be responsive to treatment with compounds in accordance with the invention, include Influenza virus infections.

Routes of Administration for Compounds in Accordance with the Invention

A medicament will usually be supplied as part of a pharmaceutical composition, which may include a pharmaceutically acceptable carrier. This pharmaceutical composition will generally be provided in a sterile form. It may be provided in unit dosage form. It will generally be provided in a sealed container, and can be provided as part of a kit. Such a kit is within the scope of the present invention. It would normally (although not necessarily) include instructions for use. A plurality of unit dosage forms may be provided.

Pharmaceutical compositions within the scope of the present invention may include one or more of the following: preserving agents, solubilising agents, stabilising agents, wetting agents, emulsifiers, sweeteners, colourants, odourants, salts (compounds of the present invention may themselves be provided in the form of a pharmaceutically acceptable salt, as explained in greater detail below), buffers, coating agents or antioxidants. They may also contain other therapeutically active agents in addition to a compound of the present invention.

Compounds of the present invention may themselves be provided in any suitable form, i.e. they may be used as such or may be used in the form of a pharmaceutically effective derivative. For example they may be used in the form of a pharmaceutically acceptable salt or hydrate. Pharmaceutically acceptable salts include alkali metal salts (e.g. sodium or potassium salts), alkaline earth metal salts (e.g. calcium or magnesium salts) aluminium salts, zinc salts, ammonium salts (e.g. tetra-alkyl ammonium salts), etc. Inorganic acid addition salts (e.g. hydrochlorides, sulphates, or phosphates) or organic acid addition salts (e.g. citrates, maleates, fumarates, succinates, lactates, propionates or tartrates) may be used.

Pharmaceutical compositions of the present invention may be provided in controlled release form. This can be achieved by providing a pharmaceutically active agent in association with a substance that degrades under physiological conditions in a predetermined manner. Degradation may be enzymatic or may be pH-dependent.

Pharmaceutical compositions may be designed to pass across the blood brain barrier (BBB). For example, a carrier such as a fatty acid, inositol or cholestrol may be selected that is able to penetrate the BBB. The carrier may be a substance that enters the brain through a specific transport system in brain endothelial cells, such as insulin-like growth factor I or II. The carrier may be coupled to the active agent or may contain/be in admixture with the active agent. Liposomes can be used to cross the BBB. WO91/04014 describes a liposome delivery system in which an active agent can be encapsulated/embedded and in which molecules that are normally transported across the BBB (e.g. insulin or insulin-like growth factor I or II) are present on the liposome outer surface. Liposome delivery systems are also discussed in U.S. Pat. No. 4,704,355.

A pharmaceutical composition within the scope of the present invention may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) routes. Such a composition may be prepared by any method known in the art of pharmacy, for example by admixing one or more active ingredients with a suitable carrier. In preferred embodiments, compounds in accordance with the invention are formulated into oral dosage forms and, therefore, are preferably provided in tablet or capsule form.

Different drug delivery systems can be used to administer pharmaceutical compositions of the present invention, depending upon the desired route of administration. Drug delivery systems are described, for example, by Langer (Science 249, 1527-1533 (1991)) and Illum and Davis (Current Opinions in Biotechnology 2m 254-259 (1991)). Different routes of administration for drug delivery will now be considered in greater detail.

(i) Oral Administration

Pharmaceutical compositions adapted for oral administration may be provided as capsules or tablets as powders or granules; as solutions, syrups or suspensions (in aqueous or non-aqueous liquids); as edible foams or whips; or as emulsions. Tablets or hard gelatine capsules may comprise lactose, maize starch or derivatives thereof, stearic acid or salts thereof. Soft gelatine capsules may comprise vegetable oils, waxes, fats, semi-solid, or liquid polyols etc. Solutions and syrups may comprise water, polyols and sugars. For the preparation of suspensions, oils (e.g. vegetable oils) may be used to provide oil-in-water or water-in-oil suspensions.

An active agent intended for oral administration may be coated with or admixed with a material that delays integration and/or absorption of the active agent in the gastrointestinal tract (e.g. glyceryl monostearate or glyceryl distearate may be used).

Thus, the sustained release of an active agent may be achieved over many hours and, if necessary, the active agent can be protected from being degraded within the stomach. Pharmaceutical compositions for oral administration may-be formulated to facilitate release of an active agent at a particular gastrointestinal location due to specific pH or enzymatic conditions.

(ii) Transdermal Administration

Pharmaceutical compositions adapted for transdermal administration may be provided as discrete patches intended to remain in intimate contact with the epidermis of the recipient for a prolonged period of time. For example, the active ingredient may be delivered from the patch by iontophoresis. (Iontophoresis is described in Pharmaceutical Research, 3(6):318 (1986).

(iii) Topical Administration

Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. Here the active ingredient can be dissolved or suspended in a suitable carrier, e.g. in an aqueous solvent. Pharmaceutical compositions adapted for topical administration in the mouth include lozenges, pastilles and mouthwashes.

(iv) Rectal Administration

Pharmaceutical compositions adapted for rectal administration may be provided as suppositories or enemas.

(v) Nasal Administration

This includes not only administration to the nasal cavity, but also administration via the nasal cavity to another location, e.g. to the lungs.

Pharmaceutical compositions adapted for nasal administration may use solid carriers, e.g. powders (preferably having a particle size in the range of 20 to 500 microns). Powders can be administered in the manner in which snuff is taken, i.e. by rapid inhalation through the nose from a container of powder held close to the nose. Compositions adopted for nasal administration may alternatively use liquid carriers, e.g. include nasal sprays or nasal drops. These may comprise aqueous or oil solutions of the active ingredient.

Compositions for administration by inhalation may be supplied in specially adapted devices, e.g. in pressurised aerosols, nebulizers or insufflators. These devices can be constructed so as to provide predetermined dosages of the active ingredient.

(vi) Vaginal Administration

Pharmaceutical compositions adapted for vaginal administration may be provided as pessaries, tampons, creams, gels, pastes, foams or spray formulations.

(vii) Parenteral Administration

Pharmaceutical compositions adapted for parenteral administration include aqueous and non-aqueous sterile injectable solutions or suspensions. These may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilised) condition requiring only the addition of a sterile liquid carrier, e.g. sterile water for injections, immediately prior to use. Extemporaneous injection solutions and suspensions may be prepared from sterile powders, granules and tablets.

From the above description it will be appreciated that compositions of the present invention can be formulated in many different way.

Dosages

Dosages of a compound of the present invention can vary between wide limits, depending upon the nature of the treatment, the age and condition of the individual to be treated, etc. and physician will ultimately determine appropriate dosages to be used.

However, without being bound by any particular dosages, a daily dosage of a compound of the present invention of from 10 μg to 100 mg/kg body weight may be suitable.

More preferably the dosage is from 5 to 50 mg/kg body weight/day. The dosage may be repeated as often as appropriate. If side effects develop, the amount and/or frequency of the dosage can be reduced, in accordance with good clinical practice.

Research Uses

Compounds of the present invention are useful in research. For example, they can be used as research tools for the analysis of one or more of the following: HSF, NF-κB, the heat shock response, viral replication, viral-mediated disorders, bacterial-mediated disorders, disorders mediated by radiation (e.g. by UV-radiation), inflammatory disorders, disorders of the immune system, ischemia, arterioclerosis, disorders involving cell proliferation (e.g. cancers), disorders involving damage to, or killing of cells (e.g. oxidative cell damage), and diabetes.

Other Uses

Compounds of the present invention can also be useful in treating plant viral disorders. Given that the basic mechanism of the heat shock response are believed to operate in a similar fashion in plants and animals and that it is reasonable to expect that direct antiviral effects will be produced by the compounds of invention in a similar fashion in plants and animals, the use of compounds of the present invention in treating viral infections of plants is within the scope of the present invention. These infections include, but are not limited to, infections by plants of geminiviruses, rhabdoviruses, caulimoviruses, bromoviruses, tobramoviruses, potyviruses and potexviruses. The use of compounds of the present invention in treating infections by viroids (including, but not limited to, potato spindle tumour viroid, hop stunt viroid, and coconut cadang-cadang viroid) is also within the scope of the invention.

Compounds of the present invention may be particularly useful in treating viral and other disorders affecting aquatic organisms (e.g. fish, crustaceans. etc.). Such disorders include disorders mediated by the snout ulcer virus, iridovirus, lymphocystis disease virus, infectious salmon anaemia, nodaviruses etc.

Compounds of the present invention may therefore be used in aquaculture. They may be used in food for aquatic organisms. Such food is within the scope of the present invention. It will generally be sold in sealed containers and labelled appropriately (e.g. as fish food, food for crustaceans, food for aquatic organisms, etc.). Alternatively, compounds of the present invention may be used for water treatment or for direct application to aquatic organisms. Such compounds do not therefore need to be present in foodstuffs in order to be useful in aquaculture.

### EXAMPLES

Compounds of formula II can be prepared from the equivalent compounds of formula I using the following general method (general method A).

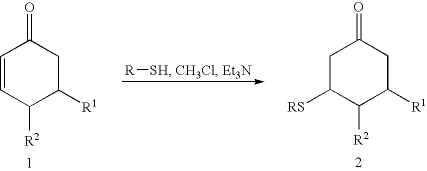

General Procedure: Add a catalytic amount of triethyl amine (20 μl) to a solution of the enone (1) (0.25 mM) and thiol (0.25-0.275 mM) in dry chloroform (5 ml), at room temperature, and stir the reaction mixture at room temperature for 1-3 days under a nitrogen atmosphere. The chloroform should then be removed under vacuum and residue purified by flash column chromatography over silica using ethyl acetate in hexane as eluent to afford the title compound 2. An example of the application of this general method is given in Example 3 below.

### Example 1

### Synthesis of 4-tert-butyldimethylsilyloxy-cyclohex-2-en-1-one (Compound A)

The subject compound was synthesised following the procedure described in J. Amer. Chem. Soc.; (1989); 111; 7; 2599-2604; Danishefsky, Samuel J.; Simoneau, Bruno and Tetrahedron Lett.; (1996); 37; 27; 4679-4682; Pour, Milan; Negishi, Ei-ichi. The reaction scheme used was as follows:-

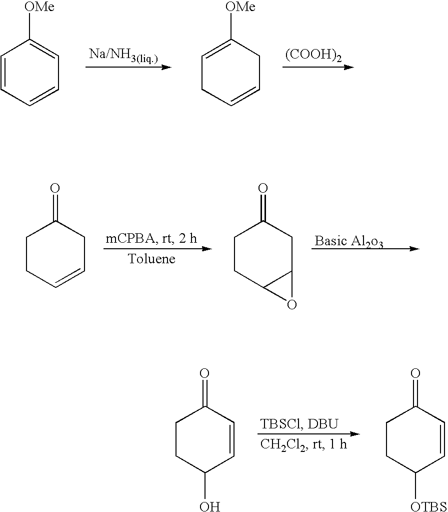

### Example 2

### Synthesis of the 5-tert-butyldimethylsilyloxy-cyclohex-2-en-1-one (Compound B)

Synthetic pathway:

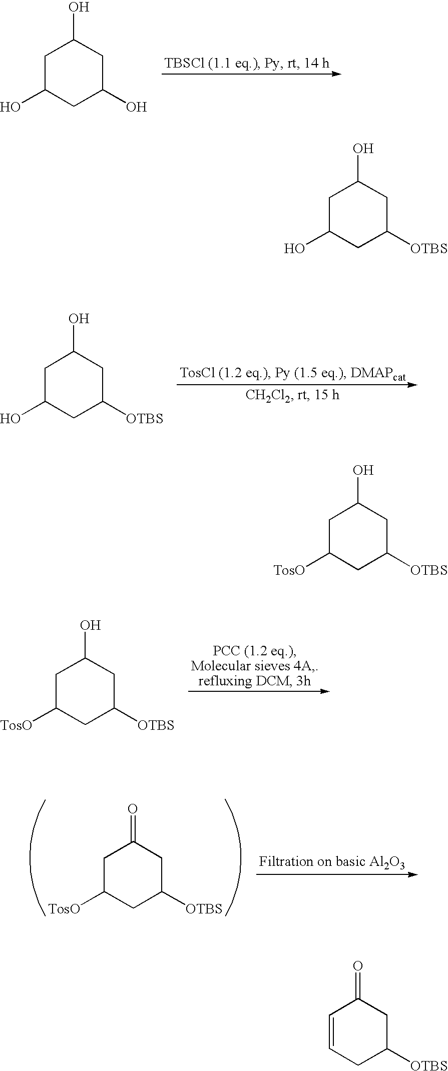

### A) Preparation of cis,cis-1,3-Dihydroxy-5-tert-butyldimethylsilyloxy-cyclohexane 2

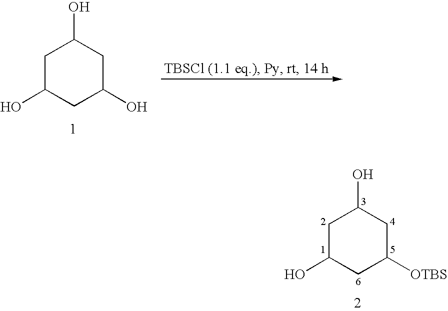

Commercially available cis,cis-1,3,5-trihydroxycyclohexane dihydrate (Aldrich: 3.02 g, 18.0 mmol) was dissolved in 150 mL of 2 mixture of anhydrous ethanol and toluene (1/1). The 2 molecules of water initially present in the starting material were removed through an azeotropic evaporation of the solvent on a rotary evaporator. This operation was repeated a second time with 34 mL of pyridine (freshly distilled and kept over KOH) as a solvent. The resulting white powder 1 was then dissolved in 45 mL of pyridine and treated with 15 mL of pre-activated molecular sieves 4A for 30 min.

The resulting anhydrous solution was then transferred with a syringe to a reaction flask; the molecular sieves were washed twice with a total amount of 30 mL of pyridine, which was combined with the first 45 mL in the reaction flask. A solution of tert-butyldimethylchorosilane (3.01 g. 19.8 mol, 1.1 eq.) in 10 mL of anhydrous THF was then added to the flask, and the reaction mixture was stirred under argon at room temperature for 14 h. The reaction was then quenched by the addition of 1 mL of water, the pyridine was removed on a rotary evaporator (below 45 ° C.) and the residue was dissolved in a mixture of ethyl acetate and water. The aqueous phase was extracted with EtOAc (3*30 mL), and the combined organic phases were washed with a saturated solution of NH₄Cl. The organic solution was then dried over MgSO₄, filtered and evaporated to yield 5.14 g of a colourless oil. The compound was then purified by CC (diethyl ether first, then EtOAc), to yield 2.17 g of the desired product (8.8 mmol, η=49%) as a white solid, along with 1.6 g of disilylated compound.

¹H-NMR (CDCl₃, 250 MHz): δ=3.84 (3 H, m, CH—O); 2.06 (3 H, m); 1.68-1.45 (5 H, m); 0.89 (9 H, s, —OSiMe₂^{t}Bu); 0.08 (6 H, s, —OSiMe₂^{t}Bu).

¹³C-NMR (CDCl₃, 400 MHz): δ=67.16, C(5); 66.29, C(1)+C(3); 42.84, C(4)+C(6); 42.67, C(2); 31.50, C(—OSiMe₂C(CH₃)₃); 25.80 C(—OSiMe₂C(CH₃)₃); −4.79, —OSiMe₂^{t}Bu).

HR-MS measured by Chemical Ionisation on [M+H⁺]: C₁₂H₂₇SiO₂
Theory: 247.17294
Found 247.17337

### B) Preparation of cis,cis-1-hydroxy-3-paratoluenesulfonate-5-tert-butyldimethylsilyloxy-cyclohexane 3

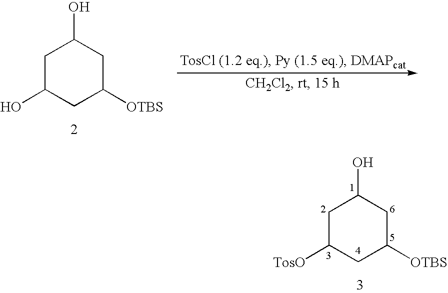

0.6 mL of dry pyridine (7.5 mmol, 1.5 eq.) and 20 mg of 4-N,N-dimethyl-pyridine (cat.) were added to a solution of cis,cis-1,3-Dihydroxy-5-tert-butyldimethylsilyloxycyclohexane (2) (1.23 g, 5.0 mmol) in 10 mL of anhydrous CH₂Cl₂. 1.14 g of paratoluene-sulfonyl chloride (6.0 mmol, 1.2 eq.) was added and the mixture was allowed to react at room temperature for 15 h under argon. At the end of this period, TLC analysis of an extracted aliquot showed no unreacted starting material 2 remaining (TLC: Et₂O/Hexane (1/1)). The reaction was then quenched by the addition of 20 mL of water, and the aqueous phase was extracted with diethyl ether (3*20 mL). The combined organic phases were washed with 30 mL of a saturated solution of NH₄Cl, dried over MgSO₄, filtered and evaporated to yield an orange oil.

The compound was then purified by CC (Et₂O/Hexane (1/1): R_{f}=0.35), to yield 1.32 g of the desired product 3 (3;3 mmol, η=66%) as a white wax.

¹H-NMR (CDCl₃, 260 MHz): δ=7.79 (2 H, d, J=8.2 Hz, Tos); 7.34 (2 H, d, J=8.2 Hz, Tos); 4.43 (1 H, m, CH—OTos); 3.59 (2 H, m, CH—OH+CH—OTBS); 2.44 (3 H, s, Tos); 2.21-1.93 (3 H, m); 1.58-1.25 (4 H, m); 0.84 (9 H, s, —OSiMe₂^{t}Bu); 0.01 & −0.01 (2*3 H, s, —OSiMe₂^{t}Bu).

¹³C-NMR (CDCl₃, 400 MHz): δ=144.74, —OSO₂—C_{tolyl}; 134.41, C_{tolyl}—CH₃; 129.86 & 127.71, CH_{tosyl}; 75.81, C(3); 65.80, C(1); 65.28 C(5); 43.53, C(6); 40.93, C(2); 40.60, C(4); 30.90, C(—OSiMe₂C(CH₃)₃); 25.72, C(—OSiMe₂C(CH₃)₃); 21.63, Me_{tosyl}; 4.81, —OSiMe₂^{t}Bu).

HR-MS: Chemical ionisation (NH,); [M+H⁺: C₁₉H₃₃SiSO₅]: theory: 401.18182
found: 401.18110

Microanalysis theory: C, 56.97; H, 8.05; S, 8.00; Si, 7.01
found: C, 56.68; H, 8.04; others not measured.

### C) Preparation of 5-tert-butyldimethylsilyloxy-cyclohex-2-en-1-one 4

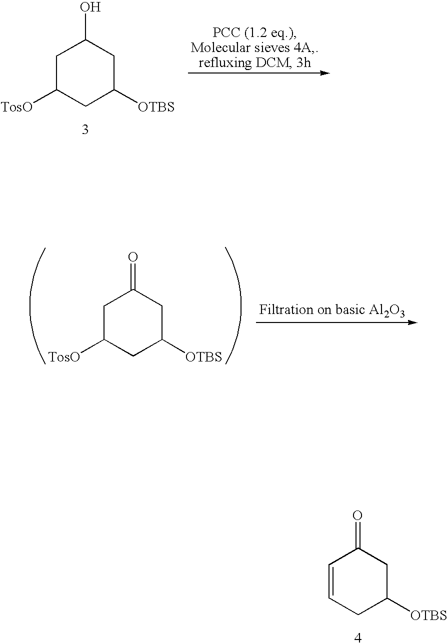

0.79 g of pytidinium chlorochromate PCC (3.68 mmol, 1.2 eq.) was added to a solution of cis,cis-1-hydroxy-3-paratoluenesulfonate-5-tert-butyldimethyl-silyloxy-cyclohexane (3) (1.23 g, 3.07 mmol) in 20 mL of dry CH₂Cl₂ at zoom temperature under argon. The suspension was then heated to reflux for 3 h. At the end of this period, TLC analysis of an extracted aliquot showed no unreacted starting material 3 remaining and two new compounds to have been formed; an oxidation product (R_{f}=0.1) and the subject compound 4 itself (R_{f}=0.33) (TLC: Et₂O/Hexane (1/3)). After the reaction had been cooled down to room temperature, the suspension was filtered through a 5 cm high basic aluminium oxide pad (column), with diethyl ether as an eluent. The filtrate, containing compound 4 was then evaporated to yield 580 mg of a colourless oil. The compound was then further purified by CC (Et₂O/Hexane (1/3): R_{f}=0.33), to yield 556 mg of 4 (2.45 mmol, η=80%) as colourless oil, which crystallizes in the freezer into a white solid (Mp<0 ° C.).

¹H-NMR (CDCl₃, 250 MHz): δ=6.88 (1 H, ddd, J(2,3)=12.7, J(3,4)+J(3,4′)=6.5 & 4.5 Hz, H-C(3)). 6.06 (1 H, dt, J(2,4)=J(2,4′)=2.4 Hz, H-C(2)); 4.24 (1 H, ddt, J(5,6)=11.4, J(4,5)=9.3 & J(5,6′)=J(5,4′)=5.5 Hz, H-C(5)); 2.67 & 2.48 (2 H, System ABX, J_{gem}=19.2, H+H′—C(6)); 2.60 & 2.38 (2 H, System ABX₂Y, H+H′-C(4)); 0.89 (9 H, s. —OSiMe₂^{t}Bu): 0.07 (6 H, s, —OSiMe₂^{t}Bu).

¹³C-NMR (CDCl₃, 400 MHz): δ=147.13, C(3); 130.52, C(2); 67.99, C(5); 48.44, C(6); 35.96, C(4); 26.07, C(—OSiMe₂C(CH₃)₃); −4.38 & −4.46, —OSiMe₂^{t}Bu). (Quaternary carbons are missing . . . to be done again)

HR-MS: Chemical ionisation (NH₃); [M+H⁺: C₁₂H₂₃SiO₂]: theory: 227.14673
found: 227.14672

### Example 3

### Preparation of (R)-2-tert-Butoxycarbonylamino-3-[(1S,2S)-2-(tert-butyl-dimethyl-silanyloxy)-5-oxo-cyclohexylsulfanyl]-propionic acid methyl ester

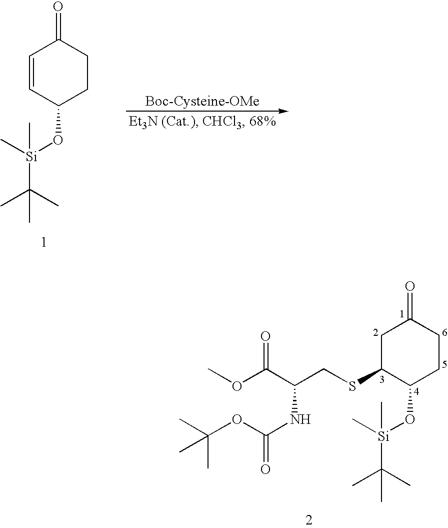

Following general method A, a solution of Boc-cysteine (60 mg, 0.25 mmol) with a catalytic amount of triethylamine (3 drops) in anhydrous chloroform (1.4 cm³) was added to a solution of enone 1 (56 mg. 0.25 mmol) in anhydrous chloroform (1 cm³). The reaction was stirred under Argon for 16 hours. TLC analysis confirmed the disappearance of the enone. The solvent was removed under reduced pressure giving a colourless oil. Purification by flash column chromatography [R_{f}=0.30 (diethyl ether/hexane; 1:1)) gave the adduct 2 (78 mg, 68% yield) as a colourless oil which solidified on standing at r.t.; the cysteine adduct is then recrystallised from (diethyl ether/hexane; 1/3); m.p. 91-93° C.; [α]_{D}²⁰+62 (c=1.0, CHCl₃); δ_{H} (250 MHz, CDCl₃) 0.12 (6H, br. s, —Si^{t}Bu(CH₃)₂), 0.91 (9H, s, —SiMe₂C(CH₃)), 1.44 (9H, s, —OC(CH₃)₃), 1.80-1.90 (1H, m, AB-C(5)), 2.12-2.30 (2H, m, AB-C(5)+AB-C(6)), 2.37 (1H, dd, J 15.0 and 3.0 Hz, AB-C(2)), 2.52-2.68 (1H, m, AB-C(6)), 2.98 & 3.00 (2H, br. AB, —CH₂S—), 3.05 (1H, dd, J 5.0 and 15.0 Hz, AB-C(2)), 3.17 (1H, m, H—C(3)), 3.74 (3H, s, —OCH₃), 4.02 (1H, m, H—C(4)), 4.51-4.60 (1H, m, CH(Cys)), 5.36 (1H, d, J 8.0 Hz, NH).

Activity of Compounds in Accordance with the Invention

Preferred compounds of the present invention have activity in one or more of the assays described in Examples 4 and 5 below.

### Example 4

### Effect of Inventive Compounds on the Reactivity of Transcription Factors HSF and NF-κB

Methods: Human lympbobiastoid Jurkat T cells were grown at 37° C. in a 5% CO₂ atmosphere in RPM1 1640 medium (GIBCO BRL, Gaithersburg, Md.) supplemented with 10% fetal calf serum (FCS, Hyclone Europe Ltd, UK) 2 nM glutamine and antibiotics according to the method described by A. Rossi et al. (Proc. Natl. Acad. Sci. USA 94: 746-750, 1997). The test compounds were stored as a 100% ethanolic stock solution (100 mM) or in DMSO (100 mM) and diluted to the appropriate concentration in culture medium at the time of use. Cells were treated with different concentrations of test compound for 1 hour and then stimulated with 12-O-tetradecanoylphorbol-13-acetate (TPA, 25 ng/ml), which is a strong inducer of NF-κB. Control cells received an equal amount of control diluent. After 3 hours whole-cell extracts were prepared and subjected to analysis of DNA-binding activity by EMSA (Electrophoretic Mobility Shift Assay) for detection of HSF or NF-κB activation, according to the method described by A. Rossi et al. (Proc. Natl. Acad. Sci. USA 94: 746-750, 1997).

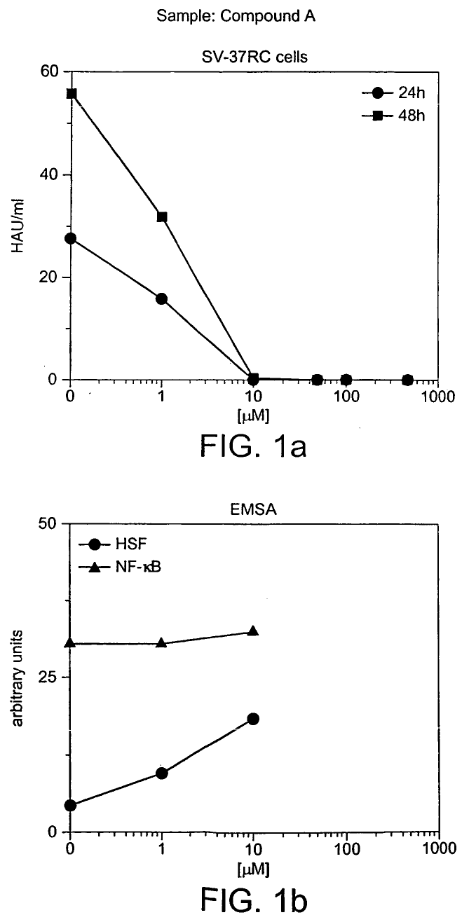

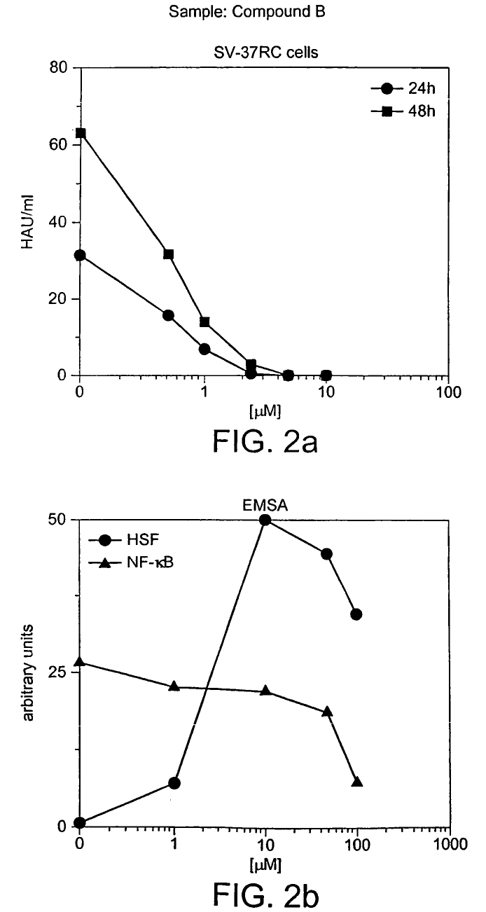

Specificity of protein-DNA complexes was verified by immunoreactivity with polyclonal antibodies specific for p65 (Rel A) or for HSF-1, for NF-κB and HSF respectively. Quantitative evaluation of NF-κB- and HSF-DNA complex formation was determined by Molecular Dynamics Phosphorlmager (MDP) analysis and was expressed in arbitrary units, as described in A. Rossi et al. (J. Biol. Chem. 273: 16446-16452, 1998). The results from representative experiments are shown in FIG. 1b for compound A (as identified above) and in FIG. 2b for compound B (as identified above).

### Example 5

### Effect of Inventive Compounds on the Reactivity of Transcription Factors HSF and NF-κB (Second Method)

Method: HeLa cell clone 13B, stably transfected with a luciferase reporter plasmid controlled by the human hsp70 promoter, and HeLa κB-transformed cells, stably transfected with a luciferase reporter plasmid controlled by a synthetic NF-κB-STM construct, were maintained in DMEM medium supplemented with 10% FBS, L-glutamine (2 mM) and G418 (250 μg/ml) at 37° C. in a 5% CO₂ humidified atmosphere.

Cells were seeded at a density of 4×10⁴ cells/well in 96-well plates. After 18-20 h, the medium was removed and cells were treated for 8 h with the test compounds (100 μl) at the appropriate dilutions in serum-free medium. For the NF-κB-dependent reporter gene assay, cells were stimulated with TPA (25 ng/ml) 2h after exposure to the compounds.

After incubation, the medium was removed and cells were lysed in 10 μl of lysis buffer. The luciferase activity was determined by adding 100 μl of substrate and measuring the release of light using a Victor 1420 microplate reader (Wallac, Finland).

Production of HeLa Cell Cones Stably Transfected with N-κB-LUC

The fragment Kpn I-BamH I from the pGL3 basic vector containing the luciferase gene (PROMEGA) was inserted in the pcDNA3 vector (INVITROGEN) digested with Bgl II-KpnI. (This digestion removes the CMV-promoter from pcDNA3.) The resulting new vector was digested with Kpn I-Hind III and a promoter containing a ‘5×NF-κB binding sites—TATA box’ sequence was inserted upstream of the luciferase gene. This vector has been named STM.

To obtain stable HeLa cell lines expressing the luciferase gene under the control of NF-κB, HeLa cells were transfected (using lipofectamine plus GIBCO) with the STM-Pvu I linearized vector, and selected for 20 days with G418 (800 μg/ml). After selection, the resistant HeLa cell pool was controlled (in quadruplicate samples) for luciferase activity after stimulation with TNFα, IL-1 and TPA. The respective luciferase activities were:
1) Control: 1369±149
2) TNFα: 6111±1231
3) IL-1: 11814±1151
4) TPA: 7181±444
   Clones were selected.

The results obtained for 2-tert-Butoxycarbonylamino-3-[2-(tert-butyl-dimethyl-silanyloxy)-5-oxo-cyclohexylsulfanyl]-propionic acid methyl ester (CTM-68), a compound of formula C, and 2-tert-Butoxycarbonylamino-3-[3-(tert-butyl-dimethyl-silanyloxy)-5-oxo-cyclohexylsulfanyl]-propionic acid methyl ester (CTM-78), a compound of formula D, were as follows:

| | HSF; AC₂₀₀/μM | NF- κB; IC₅₀/μM |
|---|---|---|
| CTM-68 | 98 | 17 |
| CTM-78 | 45 | 22 |

The AC₂₀₀/μM for HSF is the concentration at which the tested compound doubled the HSF activity in this assay. The IC₅₀/μM for NF-κB is the concentration at which the tested compound halved the NF-κB activity in this assay. These results show the tested compounds to be powerful inhibitors of NF-κB and activators of HSF.

### Example 6

Alamar Blue Cytotoxicity Assay

HeLa cells were plated in 96-well microtiter plates in 100 μl culture medium (4×10⁴/well). After 20 hours, the cells were exposed to the test compounds at different dilutions and incubated for the next 8 h at 37° C. in a 5% CO₂ humidified atmosphere. After 6 h incubation, the Alamar Blue was added in an amount equal to 10% of the culture volume (10 μl). Two hours after the addition of the Alamar Blue, the fluorescence was measured using a Victor 1420 microplate reader.

The results for compounds CTM-68 and CTM-78 were as follows:-

| | LC₅₀/μM |
|---|---|
| CTM-68 | >800 |
| CTM-78 | >800 |

The LC₅₀/μM is the concentration at which the tested compound killed half the cells in this assay. These results show that the tested compounds do not become significantly cytotoxic to HeLa cells until their concentration has very considerably exceeded that at which they were shown to inhibit the activity of NF-κB end activate HSF in Example 6.

### Example 7

### Effect of Inventive Compounds on the Replication of Sendai Virus

Methods: Monkey kidney 37RC cells were grown at 37° C. under the conditions described in Example 3 for T cells. The parainfluenza Sendai virus (SV) was grown in the allantoic cavity of 10-day-old embroynated eggs. Viral titre was expressed in haemagglutinating units (HAU) per ml; haemagglutinin titration was done according to standard procedures using human 0 Rh+ erythrocytes, as described in C. Amici et al. (J. Virol. 68: 6890-6899, 1994). Confluent monolayers of 37RC cells were infected with SV virus (5 HAU/10⁵ cells) for 1 h at 37° C., and then treated with different concentrations of test compounds. Virus yield at 24 hours after infection was determined in the supernatant of infected cells by HAU titration. The results of representative experiments are shown in FIG. 1a for compound A (as identified above) and in FIG. 2a for compound B (as identified above). These results show that these latter compounds are potent inhibitors of Sendai virus replication. This assay was also performed on compounds CTM-68 and CTM-78 and the results of these experiments are set out below.

The ID₅₀ (the 50% inhibitory dose/concentration) values at 24 hours for the tested compounds are given below.

| Compound | ID₅₀/μM |
|---|---|
| A | 1 |
| B | 0.4 |
| CTM-68 | 1 |
| CTM-78 | 3 |

The anti-vital effect of compounds A and B took place at a concentration below that at which they were toxic to uninfected cells.

### Example 8

### Neutral Red Assay

Cell viability was determined using the Neutral Red assay. 37RC cells were seeded at density of 6×10⁴ cells/well in 24-well plates and incubated for 24 h. Confluent 37RC monolayers were treated with the test compounds at different dilutions for 24 h at 37° C. After incubation, the medium was removed and the cells were incubated with RPM1 medium containing 40 μg/ml Neutral Red dye (500 μl/well). After 2 h at 37° C., the monolayers were washed with phosphate-buffered saline (PBS) and then with a solution containing 1% CaCl₂ and 0.5% formaldehyde. After washing, a solution containing 1% acetic acid/50% ethanol was added to the monolayers (250 μl/well). After 10 min at room temperature, the absorbance was determined with a microplate reader (Victor 1420, Wallac) at 540 nm.

The LD₅₀ (the 50% lethal dose/concentration) values for compounds CTM-68 and CTM-78 in this assay were 9.8 μM and 18.6 μM respectively, thus confirming that the anti-viral effect (see Example 7) of these compounds took place at a concentration well below that at which they were toxic to uninfected 37RC cells.

### Example 9

### MTT Assay

Cell viability can be determined by the 3-(4,5-dimethylthiazol-2-yl)-2,5-diphenyltetrazolium bromide (MTT) assay. Uninfected A549 (7.5×10⁴ cells/well in 96 well plates) or 37RC cells (2.5×10⁴ cells/well in 96 well plates) are treated with different concentrations of inventive compound or ethanol diluent for 24 hours. After this time, 10 ml of a 0.5% MTT solution in PBS is added to the monolayers and the mixture is incubated for 2 h at 37° C. Reduced MTT (formazan) is extracted from cells by adding 100 Ill of acidic isopropanol containing 10% Triton X-100, and formazan absorbance is measured in an ELISA microplate reader at two different wavelengths (540 and 690 nm).

General Remarks

The foregoing description of the invention is merely illustrative thereof and it should therefore be appreciated that various variations and modifications can be a made without departing from the spirit or scope of the invention as set forth in the accompanying claims.

Where preferred or optional features are described in connection with particular aspects of the present invention, they shall be deemed to apply mutatis mylandis to other aspects of the invention unless the context indicates otherwise.

All documents cited herein are hereby incorporated by reference, as are any citations referred to in said documents.

References

1. Feige U, Morimoto R, Yahara I, Polia B S. Stress-inducible Cellular Responses. Birkhaüser Verlag, Basel Boston Berlin, 1996.
2. Marber M S, Walker J M, Latchman D S, Yellon D M J. Clin. Invest 93 1087-1094, 1994.
3. Feinstein D L e al J. Biol. Chben. 271, 17724-17732, 1996.
4. Amici C., Giorgi C, Rossi A, Santoro M G. J. Virol 68, 6890-6897, 1994.
5. Santoro M G, in Stress-inducible Cellular Responses. (Fiege U et al. eds, Birkhaüser Verlag, Basel Boston Berlin) pp. 337-357, 1996.
6. Santoro M G, Garaci 9, Amici C. P.N.A.S. USA 86, 8407-8411, 1989.
7. Amici C, Sistonen L, Santoro M G, Morimoto R I. P.N.A.S. USA 89, 6227-6231, 1992.
8. Santoro M G, Benedetto A. Carruba G, Garaci E, Jaffe B. Science 209, 1032-1034, 1980.
9. Santoro M G, Trends Microbiol. 5, 276-281, 1997.
10. Rozera C, Carattoli A, De Marco A, Amici C, Giorgi C, Santoro M G J. Clin. Invest. 97; 1795-1803, 1996.
11. Rossi A, Eba G, Santoro M G. P.N.A.S. USA 94, 746-750, 1997.
12. Thanos D, Maniatis T. Cell 80, 529-532, 1995.
13. Rossi A, Elia G, Santoro M G. J. Biol. Chem. 271, 32192-32196, 1996.
14. Shield M J. Pharmacol Ther. 65, 125-137, 1995.
15. Sinclair S B et al. J. Clin. Invest. 84, 1063-1067, 1989.
16. Baeuerle P A and Henkel T (1994). Function and Activation of NF-Kappa B in the Immune System. Annual Reviews of Immunology 12: 141-179.
17. Colville-Nash P R et al. (1998). Inhibition of Inducible Nitric Oxide Synthase by Peroxisome Proliferator-Activated Receptor Agonists: Correlation with Induction of Heme Oxygenase 1. Journal of Immunology 161, 978-984.
18. K. J. Stone, R. D. Little, JOC, 1984, 49, 1849-1853.
19. A. Kawamoto, H. Kosugi, H. Uda, Chem. Lett., 1972, 807-810.
20. Moriguchi I, Hirono S, Liu Q, Nakagome Y, and Matsushita Y, (1992) Simple method of calculating octanol/water partition coefficient. Chem. Pharm. Bull. 40, 127-130.
21. Lipinski C, Lombardo F, Dominy B, Feeney P, (1997) Experimental and computational approaches to estimate solubility and permeability in drug discovery and development settings. Advanced Drug Delivery Reviews 23 (1997) 3-25.
22. Kondo, M.; Oya-Ito, T.; Kumagai, T.; Osawa, T.; Uchida, K. Chem. 2001, 296, 12076-12083.
23. Silverman, R. B., In The Organic Chemistiy of Drug Design and Drug Action; Academic Press; A Harcourt Science and Technology Company: San Diego, 1992, 336-338.
24. R. J. Flanagan, Chemistry in Britain, 2002, 28.
25. Meister, A., Anderson, M., E., Ann. Rep. Biochem. 1983, 52, 711-760.

1. A compound of formula I or II: 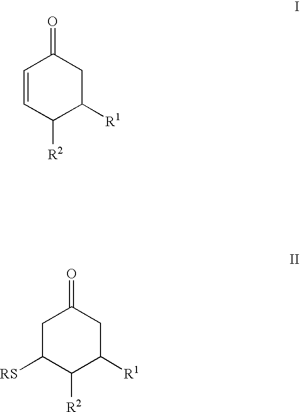
wherein:
R is a substituted or unsubstituted alkyl, alkenyl, alkynyl, aryl, aralkyl aralkenyl, or aralkynyl group, that optionally includes at least one heteroatom in its carbon skeleton; and,
R¹ and R² are H, or an —OR³ group in which R³ is a substituted or unsubstituted alkyl, alkenyl, alkynyl, aryl, aralkyl aralkenyl, or aralkynyl group containing 4-12 carbon atoms, that optionally includes at least one heteroatom in its carbon skeleton, and R¹ and R² cannot both be H:

2. A compound as claimed in claim 1 wherein R is an R⁴CH₂ group, wherein R⁴ is a substituted or unsubstituted alkyl, alkenyl, alkynyl, aryl, aralkyl aralkenyl, or aralkynyl group, that optionally includes at least one heteroatom in its carbon skeleton.

3. A compound as claimed in claim 1 or claim 2, wherein R contains 1-12 carbon atoms.

4. A compound as claimed in any of the preceding claims, wherein R includes at least one hydrophilic group.

5. A compound as claimed in claim 4, wherein said hydrophilic group is or includes a hydroxyl, carbonyl, carboxyl, amino, amido, quaternary ammonium or thiolyl group.

6. A compound as claimed in claim 5, wherein R provides the functionality of an amine, amide, peptide, ester, carboxylic acid, carboxylic acid salt, alcohol, aldehyde, ketone or thiol.

7. A compound as claimed in any of the preceding claims, wherein the group —SR is an S-cysteinyl or a substituted S-cysteinyl group.

8. A compound as claimed in claim 7, wherein the substituted S-cysteinyl group is a di- or tri-peptide group that includes an S-cysteinyl moiety.

9. A compound as claimed in claim 8, wherein the substituted S-cysteinyl group is an S-glutathionyl, S-cysteinyl, N-tert-butoxycarbonyl S-cysteinyl or N-tert-butoxycarbonyl S-cysteinyl ester group.

10. A compound as claimed in any preceding claim, wherein one of R¹ and R² is an —OR³ group and the other is H.

11. A compound as claimed in any preceding claim, wherein R³ is an alkyl group that includes a heteroatom in its carbon skeleton.

12. A compound as claimed in claim 11, wherein the heteroatom is silicon.

13. A compound as claimed in any preceding claim, wherein R³ is a trialkylsilyl group.

14. A compound as claimed in any preceding claim, wherein R³ is a tert-butyldimethylsilyl group.

15. A compound of formula II, as claimed in any preceding claim, having a calculated or measured logP value that is at least 0.25, 0.5, 0.75, 1 or 1.25 lower than the logP value for the equivalent compound of formula I, wherein the logP values for said compounds are calculated or measured using the same technique.

16. A compound as claimed in any preceding claim, that is pharmaceutically, or therapeutically active.

17. A compound as claimed in any preceding claim for use in medicine.

18. A compound as claimed in any preceding claim, for treating the human or animal body by therapy, or for use in a diagnostic method practiced upon the human or animal body.

19. A compound as claimed in any of the preceding claims having activity in respect of one or more of the following:
a) activating HSF
b) inhibiting NF-κB
c) inhibiting the replication of HSV-1
d) inhibiting the replication of Sendai virus.

20. A compound as claimed in any of claims 16-19, for treating a viral-mediated disorder, a bacterial-mediated disorder, a disorder mediated by radiation, an inflammatory disorder, a disorder of the immune system, ischemia, arteriosclerosis, a disorder involving cell proliferation, a disorder involving damage to cells or killing of cells, diabetes, a disorder affecting an aquatic organism, oxidative stress, a degenerative disease, burns or a disorder involving calcium loss or deficiency, or for use as an anti-oxidant, in combating the effects of ageing, or in promoting wound healing.

21. A compound as claimed in claim 20, wherein the disorder involving cell proliferation is a cancer.

22. A compound as claimed in claim 20, wherein the degenerative disease is neuro-degenerative disease, optionally BSE, new variant CJD, or Alzheimer's disease.

23. Use of a compound as claimed in any of claims 1 to 16 as a research tool for the analysis of one or more of the following: HSF, NF-κB, the heat shock response, viral replication, viral-mediated disorders, bacterial-mediated disorders, disorders mediated by radiation, inflammatory disorders, disorders of the immune system, damage to, or killing of cells, or diabetes.

24. A pharmaceutical composition comprising a compound according to any of claims 1-22 and optionally including a pharmaceutically acceptable carrier.

25. A composition as claimed in claim 24 for use in medicine, preferably for treating a disorder as recited in any one of claims 20-22.

26. A food for an aquatic organism comprising a compound according to any of claims 1-21.

27. An aquatic environment comprising a compound according to any of claims 1-21.

28. Use of a compound as claimed in any of claims 1-22 for the manufacture of a medicament for use in a therapeutic or diagnostic method practiced on the human or animal body.

29. A use as claimed in claim 28, for the preparation of a medicament for treating a viral-mediated disorder, a bacterial-mediated disorder, a disorder mediated by radiation, an inflammatory disorder, a disorder of the immune system, ischemia, arteriosclerosis, a disorder involving cell proliferation, cancer, a disorder involving damage to cells or killing of cells, diabetes, oxidative stress, a degenerative disease, burns, a disorder involving calcium loss or deficiency, or a disorder effecting an aquatic organism.

30. A use as claimed in claim 28, for the preparation of a medicament for use as an anti-oxidant, in promoting wound healing or use in combating the effects of ageing.

31. A use as claimed in claim 29, for the preparation of a medicament for use in treating a neuro-degenerative disease, preferably BSE, new variant CJD, or Alzheimer's disease

32. A method for treating a condition, disorder or infection in a human or animal subject, comprising administering a therapeutically effective amount of a compound as claimed in any of claims 1-22 or a composition as claimed in claim 24 or 25 to said subject.

33. A method of treating a viral-mediated disorder, a bacterial-mediated disorder, a disorder mediated by radiation, an inflammatory disorder, a disorder of the immune system, ischemia, arteriosclerosis, a disorder involving cell proliferation, cancer, a disorder involving damage to cells or killing of cells, diabetes, oxidative stress, a degenerative disease, the effects of ageing, burns, a disorder involving calcium loss or deficiency, or a disorder effecting an aquatic organism, comprising administering a compound as claimed in any one of claims 1-22 or a composition as claimed in claim 24 or 25 to a subject suffering from one or more of said conditions, in an amount effective to at least ameliorate at least one of said conditions.

34. A method of promoting wound healing, comprising administering a compound as claimed in any one of claims 1-22 or a composition as claimed in claim 24 or 25 to a wounded subject in an amount effective to promote wound healing.

35. A method as claimed in claim 33, wherein the degenerative disease is a neuro-degenerative disease, preferably BSE, new variant CJD, or Alzheimer's disease.

36. A method of decreasing the lipophilicity and/or increasing the water solubility and/or the therapeutic index of a pharmaceutically active compound of formula I as defined in any of claims 1-15, said method comprising forming an adduct of said compound of formula I and a thiol of the formula HSR, wherein R is as defined in any of claims 1-15.

37. A method as claimed in claim 36, wherein the adduct is formed via a Michael reaction between the unsaturated compound of formula I and the thiol.

38. An adduct, prepared or preparable by a method as claimed in claim 36 or claim 37.

39. A compound as claimed in any of claims 1-22, that exhibits a capacity to activate HSF at a concentration at which said compound has no significant inhibitory effect on NF-κB activity.

40. A compound as claimed in claim 39, for use in treating a condition responsive to a heat shock response.

41. A compound as claimed in claim 39 or 40, for use in treating a virally mediated disorder.

42. A compound as claimed in claim 41, for use in treating a viral infection wherein an inflammatory component is not essential to the pathology of the infecting virus, or wherein a pathological effect of the infecting virus can be reversed or prevented by a beat shock response.

43. A compound as claimed in claim 41, for use in treating an infection with a virus that is not dependant upon NF-κB for replication, or does not have κB elements in its genome.

44. A compound as claimed in claim 39 or 40, for use in a cytoprotective treatment.

45. A compound as claimed in claim 39 or 40, for use in treating a disorder that involves damaging or killing cells.

46. Use of a compound as claimed in claim 39 for the manufacture of a medicament for use in a therapeutic or diagnostic method practised on the human or animal body.

47. A use as claimed in claim 46, wherein the medicament is for use in a therapeutic treatment as defined in any one of claims 40-45.

48. A method of treating a condition, disorder or infection in a human or animal subject, comprising administering a therapeutically effective amount of a compound as claimed in claim 39 to said subject, wherein said condition, disorder or infection is as defined in any of claims 40-45.

49. A method of providing a cytoprotective treatment to a human or animal subject, comprising administering a therapeutically effective amount of a compound as claimed in any of claims 39-45 to said subject.

50. A cytoprotective method, comprising administering a cytoprotective amount of a compound as claimed in any of claims 39-45 to a human or animal subject.
